(19) United States
(12) Patent Application Publication
(10) Pub. No.: US 2019/0103183 A1
(43) Pub. Date: Apr. 4, 2019

(54) MEDICAL DIAGNOSTIC DEVICE AND MEDICAL DIAGNOSTIC PROGRAM

(71) Applicant: Konica Minolta, Inc., Tokyo (JP)

(72) Inventor: Takashi SAKAI, Kanagawa (JP)

(21) Appl. No.: 16/086,919

(22) PCT Filed: Mar. 23, 2017
(86) PCT No.: PCT/JP2017/011603

§ 371 Date: Sep. 20, 2018

(30) Foreign Application Priority Data
Mar. 28, 2016 (JP) 2016-063517

(51) Int. Cl. G16H 30/20; A61B 8/00; A61B 8/08

(52) U.S. Cl. CPC G16H 30/20; A61B 8/463; G16H 30/40; A61B 8/565; A61B 8/4444; A61B 8/488

(57) ABSTRACT

Provided is a medical diagnostic device communicating with an image display device which displays a medical image and an image management device which manages image data of the medical image, said medical diagnostic device comprising an input unit which accepts an operation input from an operator, and a control unit which, according to a save instruction signal which is generated on the basis of the operation input, transmits to the image management device the image data of the medical image together with identification information and causes the image management device to store the image data in association with the identification information, and transmits to the image display device the identification information and a display instruction signal for causing the image display device to display the image data and causes the image display device to acquire the image data from the image management device.

### TECHNICAL FIELD

The present invention relates to a medical diagnostic apparatus and a medical diagnostic program.

### BACKGROUND ART

A medical image management system (picture archiving and communication system (PACS)) is known which saves and manages in a medical image management apparatus (PACS server) ultrasound images taken by a modality such as an ultrasound diagnostic apparatus, an X-ray diagnostic apparatus, or a magnetic resonance imaging (MRI) apparatus in association with patient information, and displays the ultrasound image on a viewer screen of an image display terminal apparatus (PACS client) for interpretation and diagnosis by a doctor etc.

When a patient to be diagnosed is imaged by the modality, the doctor etc. naturally refers to an ultrasound image thereof on the viewer screen of the image display terminal apparatus and makes a diagnosis. However, in such a medical image management system, the ultrasound image taken by the modality is captured, saved, and managed in the medical image management apparatus. Thus, when referring to the ultrasound image, the doctor etc. needs to search and read the target ultrasound image from the medical image management apparatus, which is a great burden of operation for the doctor etc.

From such a background, for example, PTL 1 describes that a program of an image display terminal apparatus and a program of a modality are coordinated, and that when an image capture button provided on the image display terminal apparatus is pressed, ultrasound image data is captured from the modality and displayed on a viewer screen.

### CITATION LIST

### Patent Literature

### PTL 1

Japanese Patent Application Laid-Open No. 2014-119863

### SUMMARY OF INVENTION

### Technical Problem

The conventional system (PTL 1) is configured on the assumption that an operator such as a doctor or a technician (hereinafter referred to as “doctor etc.”) sitting on the side of the image display terminal apparatus (hereinafter referred to as “image display apparatus”) operates on the viewer screen of the image display apparatus to give the modality an imaging instruction and an instruction to capture the ultrasound image data.

However, in actual medical practice, for example, an ultrasound diagnostic apparatus is used for imaging by a doctor etc. after an interview by the doctor etc. in an examination room. Thus, while being on the side of the ultrasound diagnostic apparatus, the doctor etc. operates the ultrasound diagnostic apparatus while pressing an ultrasound probe of the ultrasound diagnostic apparatus against an area to be inspected of a patient. In other words, the ultrasound diagnostic apparatus operated by the doctor etc. sitting on the side of the image display apparatus may suffer from degraded operability of the ultrasound diagnostic apparatus. FIG. 13 shows an example of the ultrasound diagnostic system described above. In FIG. 13, reference sign 1 denotes an ultrasound diagnostic apparatus, reference sign 2 denotes an image display apparatus, reference sign 5 denotes an electronic medical record management apparatus, reference sign P1 denotes a doctor etc., and reference sign P2 denotes a patient.

In addition, in the conventional system (PTL 1), when the program of the image display apparatus and the program of the modality are to be coordinated, linkage setting using subject information such as patient ID is required. Thus, for a return-visit patient, subject information such as an electronic medical record is used, and for a first-visit patient, new subject information needs to be set. This gives a great burden on the doctor etc. when frequently using the ultrasound diagnostic apparatus.

An object of the present invention is to provide a medical diagnostic apparatus that can reduce a burden of operation by a doctor etc. when viewing a medical image on an image display apparatus, and a medical diagnostic program.

### Solution to Problem

A main aspect of the present invention for solving the above problem, a medical diagnostic apparatus configured to be capable of communicating with an image display apparatus that displays a medical image of an area to be inspected of a subject and an image management apparatus that manages image data of the medical image, includes: an input section that receives an operation input by an operator; and a control section that transmits the image data of the medical image and identification information to the image management apparatus and causes the image management apparatus to store the image data in association with the identification information according to a save instruction signal generated based on the operation input, and further transmits to the image display apparatus the identification information and a display instruction signal for causing the image display apparatus to display the image data of the medical image and causes the image display apparatus to obtain the image data of the medical image from the image management apparatus.

Also, an ultrasound diagnostic program to be executed by a medical diagnostic apparatus configured to be capable of communicating with an image display apparatus that displays a medical image of an area to be inspected of a subject and an image management apparatus that manages image data of the medical image, includes: a process of receiving an operation input by an operator; and a process of transmitting the image data of the medical image and identification information to the image management apparatus and causing the image management apparatus to store the image data in association with the identification information according to a save instruction signal generated based on the operation input, and further transmitting to the image display apparatus the identification information and a display instruction signal for causing the image display apparatus to display the image data of the medical image and causing the image display apparatus to obtain the image data of the medical image from the image management apparatus.

### Advantageous Effects of Invention

The medical diagnostic apparatus according to the present invention can reduce a burden of operation by a doctor etc. when viewing an ultrasound image on an image display apparatus.

### BRIEF DESCRIPTION OF DRAWINGS

FIG. 1 shows an example of an overall configuration of an ultrasound diagnostic system according to an embodiment;

FIG. 2 shows an example of a function block of an ultrasound diagnostic apparatus according to the embodiment;

FIG. 3 shows an appearance of the ultrasound diagnostic apparatus according to the embodiment;

FIG. 4 shows an example of a function block of an image display apparatus according to the embodiment;

FIG. 5 shows an example of an operation flow of the ultrasound diagnostic system according to the embodiment;

FIG. 6A, 6B, 6C show an example of a diagnostic screen displayed by the ultrasound diagnostic apparatus according to the embodiment;

FIG. 7 shows an example of an ultrasound image viewing screen displayed by the image display apparatus according to the embodiment;

FIG. 8 shows an example of a detailed operation flow of the ultrasound diagnostic apparatus according to the embodiment;

FIG. 9 shows an example of a detailed operation flow of the ultrasound diagnostic apparatus according to the embodiment;

FIG. 10 shows an example of a detailed operation flow of the image display apparatus according to the embodiment;

FIG. 11 shows an example of a detailed operation flow of an image management apparatus according to the embodiment;

FIG. 12 shows an example of an operation flow of an ultrasound diagnostic system according to a variant; and

FIG. 13 illustrates an ultrasound diagnostic system.

### DESCRIPTION OF EMBODIMENTS

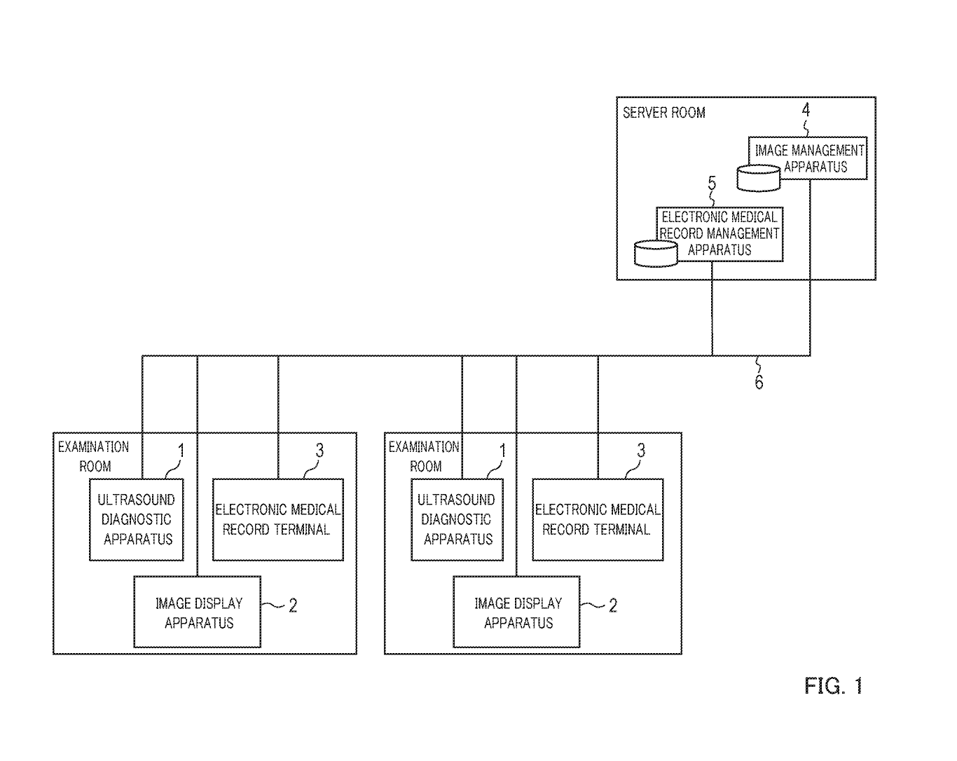

Now, an example of a configuration of an ultrasound diagnostic system according to this embodiment will be described with reference to FIGS. 1 to 4.

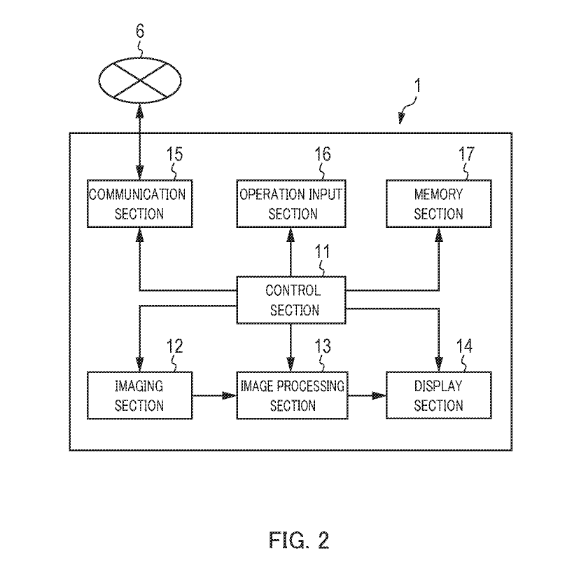

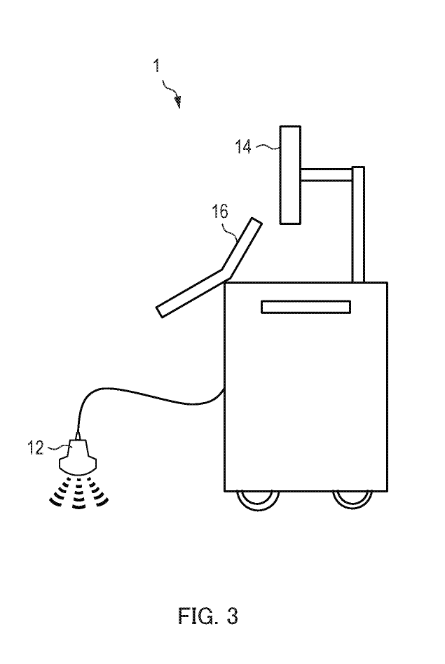

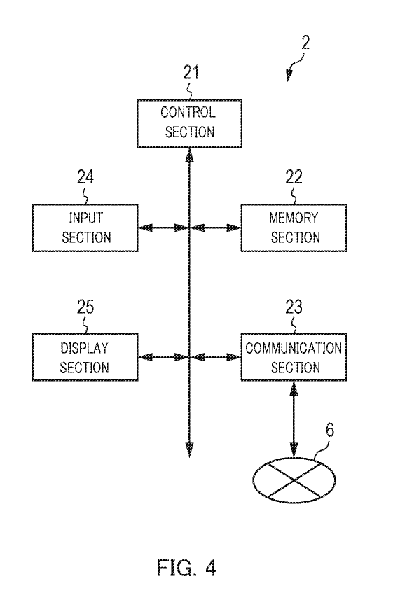

FIG. 1 shows an example of an overall configuration of the ultrasound diagnostic system. FIG. 2 shows an example of a function block of an ultrasound diagnostic apparatus. FIG. 3 shows an appearance of the ultrasound diagnostic apparatus. FIG. 4 shows an example of a function block of an image display apparatus.

The ultrasound diagnostic system according to this embodiment includes, for example, ultrasound diagnostic apparatus 1, image display apparatus 2, electronic medical record terminal 3, image management apparatus 4, electronic medical record management apparatus 5, and communication line 6 (see FIG. 1).

The ultrasound diagnostic system used in a hospital will be described here. Ultrasound diagnostic apparatus 1, image display apparatus 2, and electronic medical record terminal 3 are placed in an examination room in the hospital, image management apparatus 4 and electronic medical record management apparatus 5 are placed in a server room. These apparatuses are connected to communicate through communication line 6. Communication line 6 is, for example, a local area network (LAN) line provided in the hospital, and the apparatuses perform data communication in compliance with Digital Imaging and Communication in Medicine (DICOM) standards.

First, a flow of an examination of a patient in the hospital will be described.

In the hospital, a receipt terminal (not shown) is provided, and when the patient visits the hospital, the receipt terminal receives an electronic medical record of the patient from electronic medical record management apparatus 5. Then, a person at the reception desk checks the contents of the electronic medical record on a display screen of the receipt terminal, and transmits the electronic medical record from the receipt terminal to electronic medical record terminal 3.

In the examination room, ultrasound diagnostic apparatus 1, image display apparatus 2, and electronic medical record terminal 3 are placed, and the doctor performs an examination using ultrasound diagnostic apparatus 1, image display apparatus 2, and electronic medical record terminal 3. Electronic medical record terminal 3 stores electronic medical records of a plurality of patients to be examined in the order of receiving the electronic medical records from the receipt terminal. Then, the patients are brought into an examination waiting state, and when the doctor performs an update operation of a patient to be examined, electronic medical record terminal 3 displays the contents of an electronic medical record of a next patient to be examined on a display screen.

The doctor examines the patient to be examined, and inputs the contents of the examination in electronic medical record terminal 3. At this time, the doctor inspects the patient using ultrasound diagnostic apparatus 1 as required. Ultrasound diagnostic apparatus 1 performs the inspection and then saves image data including an obtained ultrasound image in image management apparatus 4. The image data saved in image management apparatus 4 is read by image display apparatus 2, and the ultrasound image is displayed on a display screen of image display apparatus 2. The doctor provides an explanation to the patient using the medical image displayed on the display screen of image display apparatus 2, and inputs findings in electronic medical record terminal 3. When the examination is finished and the doctor inputs an examination finish instruction to electronic medical record terminal 3, electronic medical record terminal 3 transmits the electronic medical record to the receipt terminal. Then, the doctor performs an update operation of a patient to be examined, and examines a next patient.

Ultrasound diagnostic apparatus 1 displays and outputs an ultrasound image of a state of internal body tissue of a subject (see FIGS. 2 and 3). Hereinafter, a patient to be inspected using ultrasound diagnostic apparatus 1 is referred to as “subject”, and data on the subject is also referred to as “subject information”.

Ultrasound diagnostic apparatus 1 here generates, for example, a color Doppler image as an ultrasound image. The color Doppler image is generated by displaying a color flow image expressing the blood flow and motion of body tissue such as the myocardial wall by color over a brightness mode image expressing tomogram of a living body by gray scale.

Ultrasound diagnostic apparatus 1 includes control section 11, imaging section 12, image processing section 13, display section 14, communication section 15, operation input section 16, and memory section 17 (see FIG. 2).

Control section 11 collectively controls operations of the sections in ultrasound diagnostic apparatus 1. Control section 11 is realized, for example, by a CPU (Central Processing Unit) executing a program.

Imaging section 12 images an area to be inspected of a subject, and generates two-dimensional data in frames for generating an ultrasound image.

Imaging section 12 includes an ultrasound probe constituted by a plurality of transducers, and transmits an ultrasound beam into the subject with the ultrasound probe being pressed against the abdomen or the like of the subject. The ultrasound probe receives an ultrasound echo reflected in the subject and generates a receiving signal. When generating a brightness mode image, imaging section 12 accumulates in a line memory changes with time of the intensity of the receiving signal generated by the ultrasound echo. Then, imaging section 12 scans the plurality of transducers in the ultrasound probe, switches a direction of transmitting and receiving the ultrasound beam, and sequentially accumulates the receiving signal in each scan position in the line memory to generate two-dimensional data in frames. The brightness mode image is generated by converting the intensity of the receiving signal for the two-dimensional data into a brightness value. When generating a color flow image, imaging section 12 detects ultrasound echoes of successively transmitted pulse-like ultrasound beams from the same depth position. Then, imaging section 12 calculates the speed, power, and dispersion of the blood flow in the depth position based on a phase difference between the successive ultrasound echoes to generate a blood flow signal. As is the case for the brightness mode image, imaging section 12 scans the ultrasound probe, switches a direction of transmitting and receiving the ultrasound beam, and sequentially accumulates the blood flow signal in each scan position in the line memory to generate two-dimensional data in frames. The color flow image is generated by converting the speed, power, and dispersion of the blood flow signal for the two-dimensional data into an RGB value.

Image processing section 13 obtains the two-dimensional data in frames from imaging section 12 to generate an ultrasound image. Specifically, as described above, image processing section 13 generates a brightness mode image from two-dimensional data on the intensity of the receiving signal, and generates a color flow image from the two-dimensional data on the speed, power, and dispersion of the blood flow signal. Then, image processing section 13 superimposes the moving color flow image on the brightness mode image to generate a color Doppler image.

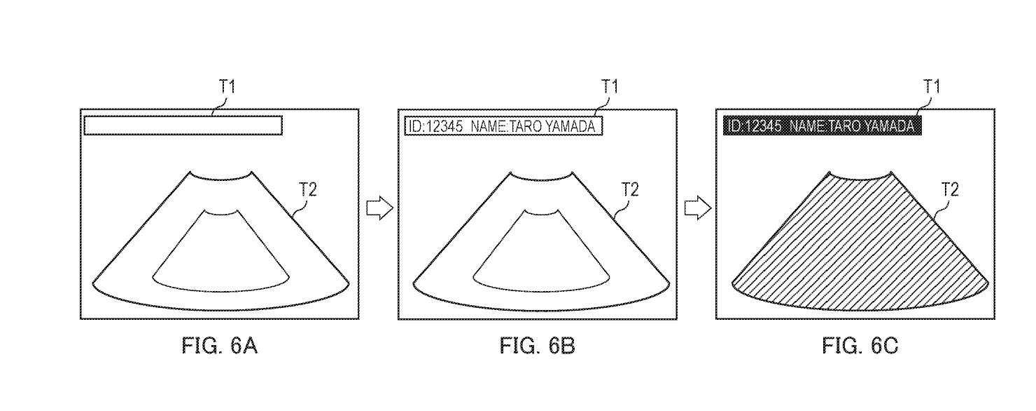

Image processing section 13 generates image data (hereinafter also referred to as “ultrasound image data”) including the ultrasound image generated in this manner and an image indicating identification information of the subject, and causes display section 14 to display the image data (described later with reference to FIG. 6). Image processing section 13 includes a buffer memory for generating the images described above, and a high-capacity memory capable of holding image data of generated images. Then, in response to a save instruction from control section 11, image processing section 13 encodes and sends the image data to memory section 17 to store therein the image data as still image data or moving image data. Image processing section 13 performs coordinate transformation or data interpolation of the ultrasound image to convert the ultrasound image into image data that can be displayed on display section 14 and output the image data.

Display section 14 is a display apparatus such as an LCD or a CRT display. Display section 14 displays the ultrasound image on the display screen based on the image data of the ultrasound image generated by image processing section 13.

Communication section 15 is a communication interface for transmitting and receiving data to and from an external device such as electronic medical record terminal 3, image management apparatus 4, and electronic medical record management apparatus 5 through communication line 6, and is constituted, for example, by a LAN adapter.

Operation input section 16 (input section) is, for example, a touch panel or an operation button, and receives an input operation by the doctor etc. Data input to operation input section 16 by the doctor etc. is output as an operation signal to control section 11. Ultrasound diagnostic apparatus 1 according to this embodiment includes, as operation input section 16, operation buttons for an imaging stop operation, imaging restart operation, still image saving operation, moving image saving start operation, moving image saving finish operation, or the like.

Memory section 17 includes, for example, ROM, RAM, HDD, or the like, and has various processing programs such as a system program or an ultrasound diagnostic program executable on the system program, various data, and a work area for temporary storage in arithmetic processing.

Memory section 17 saves the image data including the ultrasound image generated as described above. Memory section 17 can save, as the image data, for example, still image data that is image data of a still image for one frame, and moving image data generated so that still image data for several frames can be displayed as a moving image. The image data of the ultrasound image stored in memory section 17 is then transmitted to image management apparatus 4 and managed by image management apparatus 4.

Image display apparatus 2 is an information processing apparatus for viewing the ultrasound image taken by ultrasound diagnostic apparatus 1. Image display apparatus 2 obtains the image data of the ultrasound image from image management apparatus 4 and displays the image.

Image display apparatus 2 is a computer including, for example, control section 21, memory section 22, communication section 23, input section 24, and display section 25 (see FIG. 4).

Control section 21 collectively controls the sections in image display apparatus 2, and realized by a CPU executing a computer program.

Memory section 22 includes RAM, ROM, HDD, or the like, and stores a system program, an image viewing program executable on the system program, image format data used for application, or the like.

Communication section 23 is a communication interface for transmitting and receiving data to and from an external device such as ultrasound diagnostic apparatus 1 or image management apparatus 4 through communication line 6, and is constituted, for example, by a LAN adapter.

Input section 24 is a user interface used by the doctor etc. to perform an operation input to image display apparatus 2, and is constituted, for example, by a keyboard.

Display section 25 displays the ultrasound image, and is constituted, for example, by a liquid crystal display.

Electronic medical record terminal 3 is an information processing apparatus that displays information on the subject as an electronic medical record and to which the doctor etc. inputs an examination result. Electronic medical record terminal 3 is a computer like image display apparatus 2, and detailed descriptions thereof will be omitted.

Image management apparatus 4 is, for example, a server apparatus that saves and manages ultrasound image data. Image management apparatus 4 is a computer like image display apparatus 2, and detailed descriptions thereof will be omitted.

Image management apparatus 4 includes an image data base and stores the ultrasound image data and information on the image such as patient ID, image ID, or inspection information assigned thereto. The inspection information includes information on inspection such as inspection date, time of saving of an image file, depth of display, inspection ID for identifying inspection, name of inspection, acceptance number, purpose of inspection, or contents of inspection.

Electronic medical record management apparatus 5 is a server apparatus that saves and manages electronic medical records. Electronic medical record management apparatus 5 is a computer like image display apparatus 2, and detailed descriptions thereof will be omitted.

Electronic medical record management apparatus 5 includes an electronic medical record data base, and saves an electronic medical record for each patient in the electronic medical record data base. The electronic medical record includes, for example, information on a patient such as name of the patient, patient ID, sex, date of birth, address, occupation, or examination results. Part or all of the contents of data in the electronic medical record are displayed as the subject information by electronic medical record terminal 3 or ultrasound diagnostic apparatus 1.

### Operation of Ultrasound Diagnostic System

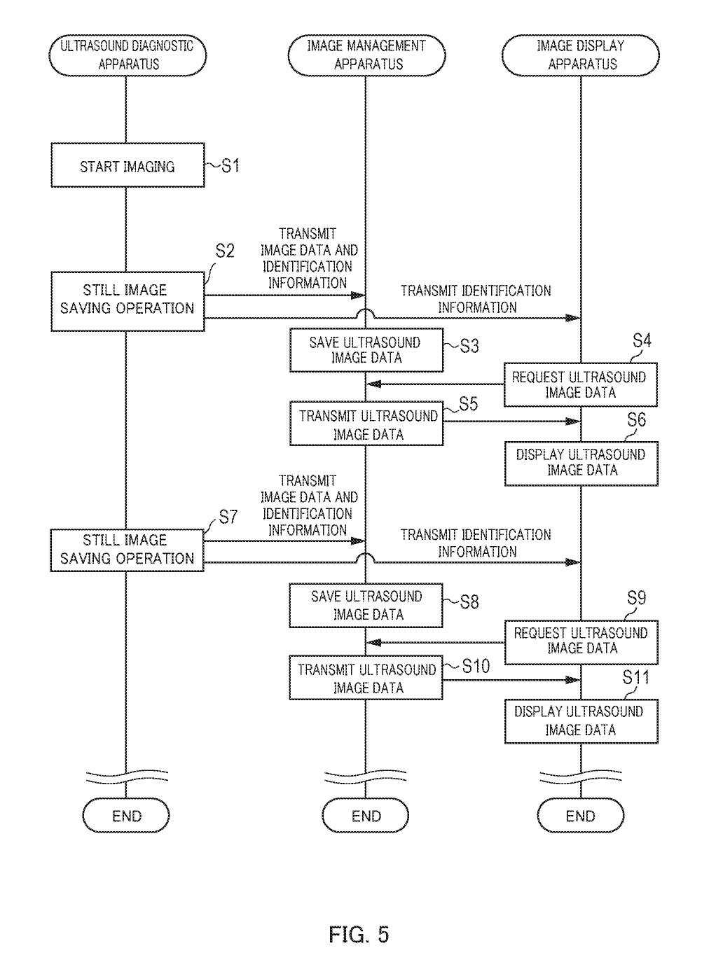

Next, with reference to FIGS. 5 to 7, an outline of an operation flow of the ultrasound diagnostic system according to this embodiment will be described.

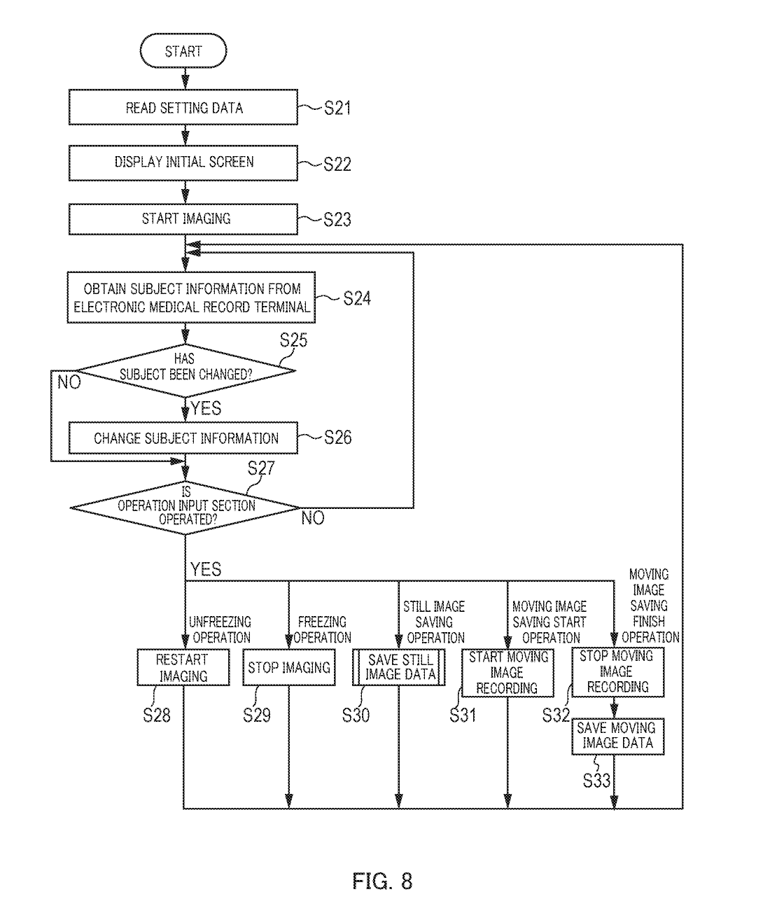

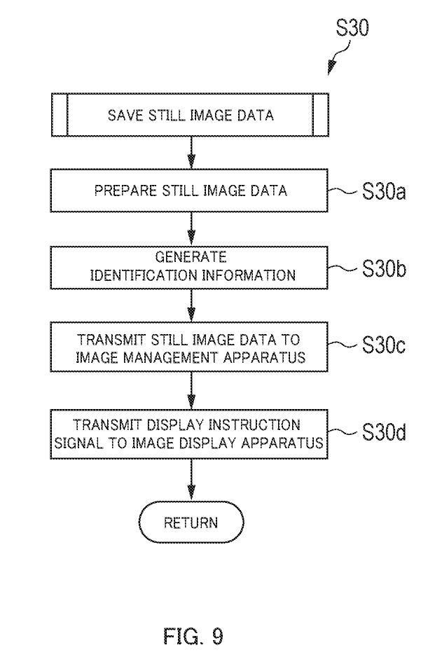

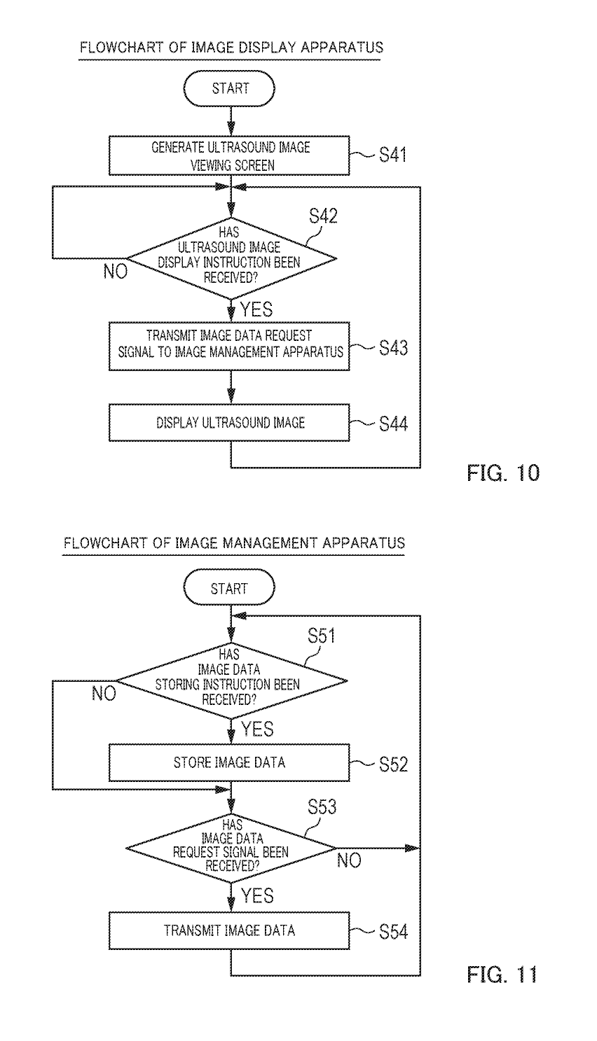

FIG. 5 shows an example of an operation flow of the ultrasound diagnostic system. A coordinating operation of image management apparatus 4 and image display apparatus 2 will be mainly described here when ultrasound diagnostic apparatus 1 performs an operation for saving an ultrasound image. This operation of the ultrasound diagnostic system is achieved, for example, by the programs of ultrasound diagnostic apparatus 1, image display apparatus 2, and image management apparatus 4 cooperating with each other. A detailed operation flow of ultrasound diagnostic apparatus 1 will be described later with reference to FIGS. 8 and 9. Detailed operation flows of image display apparatus 2 and image management apparatus 4 will be described later with reference to FIGS. 10 and 11, respectively.

FIG. 6 shows an example of a diagnostic screen displayed by ultrasound diagnostic apparatus 1. FIG. 6A shows an initial diagnostic screen, FIG. 6B shows a diagnostic screen with the subject information being set, and FIG. 6C shows a diagnostic screen when an ultrasound image is taken. The diagnostic screens in FIGS. 6A to 6C are generated by image processing section 13 of ultrasound diagnostic apparatus 1, and sequentially updated according to control by control section 11. The diagnostic screens are generated, for example, based on ultrasound image data and an image format.

In FIGS. 6A to 6C, reference sign T1 denotes a subject display region to which identification information of a subject is input, and for example, patient ID and name of the subject are displayed in the subject display region. Reference sign T2 denotes an ultrasound image display region to which an ultrasound image of an area to be inspected of the subject is input, and for example, a color Doppler image is displayed in the ultrasound image display region.

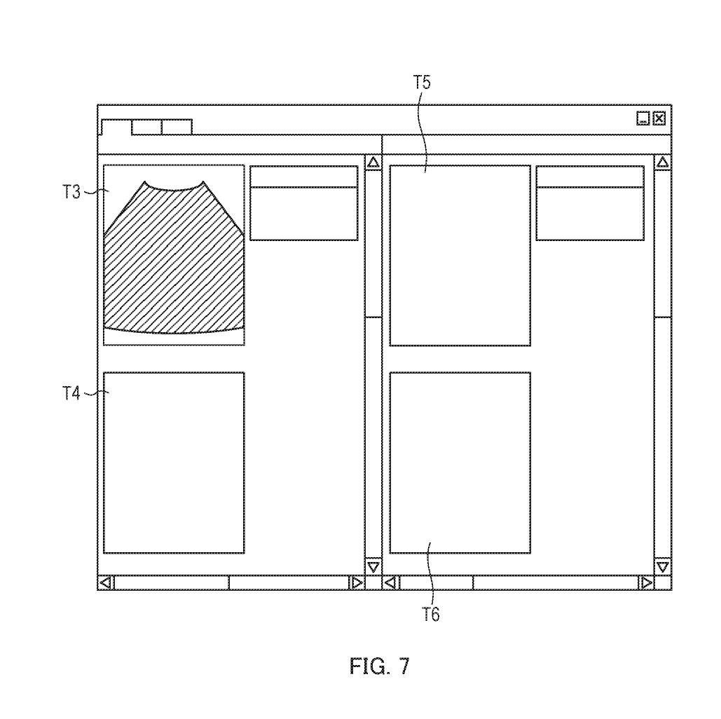

FIG. 7 shows an example of a viewing screen displayed by image display apparatus 2. The viewing screen (see FIG. 7) displayed by image display apparatus 2 has, for example, a plurality of ultrasound image display regions T3 to T6. Every time image display apparatus 2 receives ultrasound image data, image display apparatus 2 displays ultrasound images in ultrasound image display regions T3, T4, T5, and T6 in order.

First, when ultrasound diagnostic apparatus 1 is powered on to start an ultrasound diagnostic program, imaging section 12 starts imaging (step S1). When ultrasound diagnostic apparatus 1 according to this embodiment is powered on, imaging section 12 continuously performs imaging. Then, if the ultrasound probe of imaging section 12 is pressed against the area to be inspected of the subject, a taken ultrasound image is input to region T2 and displayed on the diagnostic screen of ultrasound diagnostic apparatus 1 (FIG. 6C). The image data on the diagnostic screen on which the ultrasound image and the subject information are displayed corresponds to “ultrasound image data” to be saved.

If the doctor etc. performs a still image saving operation on ultrasound diagnostic apparatus 1, still image data saving processing is performed (step S2). In this case, control section 11 first generates, for example, image ID as identification information for identifying ultrasound image data to be saved. Then, ultrasound diagnostic apparatus 1 transmits to image management apparatus 4 the ultrasound image data to which the image ID is assigned and a storing instruction signal. Further, ultrasound diagnostic apparatus 1 transmits to image display apparatus 2 a display instruction signal and the image ID so that the image of the ultrasound image data is viewed and displayed. Details of “still image data saving processing” performed by ultrasound diagnostic apparatus 1 will be described later with reference to S30 in FIG. 8 and FIG. 9.

In response to receiving the ultrasound image data and the image ID from ultrasound diagnostic apparatus 1, image management apparatus 4 stores the ultrasound image data in association with the image ID in the image data base (step S3).

In response to receiving the display instruction signal from ultrasound diagnostic apparatus 1, image display apparatus 2 transmits to image management apparatus 4 a request signal for the ultrasound image data so as to obtain the ultrasound image data from image management apparatus 4 (step S4). At this time, image display apparatus 2 transmits to image management apparatus 4 the image ID assigned to the display instruction signal and the request signal for the ultrasound image data.

In response to receiving the request signal for the ultrasound image data from image display apparatus 2, image management apparatus 4 transmits the ultrasound image data corresponding to the image ID to image display apparatus 2 (step S5).

Image display apparatus 2 waits for the ultrasound image data being transmitted from image management apparatus 4, and in response to receiving the ultrasound image data, displays an image of the ultrasound image data on the viewing screen (step S6). Here, the image of the ultrasound image data on the subject first received is displayed in ultrasound image display region T3 on the viewing screen (see FIG. 7) displayed by image display apparatus 2.

Then, when the doctor etc. again performs the still image saving operation on ultrasound diagnostic apparatus 1, still image data saving processing is performed (step S7).

Also in this case, ultrasound diagnostic apparatus 1 performs the same processing as described above. Ultrasound diagnostic apparatus 1 first generates image ID different from the above, and transmits to image management apparatus 4 the ultrasound image data to which the image ID is assigned and a storing instruction signal. Then, ultrasound diagnostic apparatus 1 transmits the image ID and a display instruction signal to image display apparatus 2.

Image management apparatus 4 and image display apparatus 2 perform processings in steps S8 to S11 as in steps S3 to S6 according to the display instruction signal. In response to receiving second ultrasound image data, image display apparatus 2 displays an image of the ultrasound image data in ultrasound image display region T4 on the viewing screen (step S11).

As such, in the ultrasound diagnostic system according to this embodiment, the ultrasound image can be displayed by image display apparatus 2 without requiring an operation on image display apparatus 2. Thus, the doctor etc. does not need to search corresponding data from image management apparatus 4 when viewing the ultrasound image on image display apparatus 2 after taking the ultrasound image using ultrasound diagnostic apparatus 1.

Next, details of operations of ultrasound diagnostic apparatus 1, image display apparatus 2, and image management apparatus 4 in the ultrasound diagnostic system according to this embodiment will be described.

### Operation of Ultrasound Diagnostic Apparatus

FIGS. 8 and 9 show an example of a detailed operation flow of ultrasound diagnostic apparatus 1. A coordinating operation of ultrasound diagnostic apparatus 1 in the ultrasound diagnostic system described above will be mainly described in connection with step S30.

Steps in FIGS. 8 and 9 are performed by control section 11 of ultrasound diagnostic apparatus 1 according to an ultrasound diagnostic program.

When the ultrasound diagnostic program is executed, control section 11 first reads setting data stored in memory section 17, and sets the setting data as variables in various processings (step S21).

The setting data includes, for example, address data, apparatus setting data, display setting data, subject setting data, or the like of image display apparatus 2 and image management apparatus 4 to be coordinated. The address data of image display apparatus 2 and image management apparatus 4 to be coordinated are set, for example, as IP addresses. Control section 11 transmits a display instruction signal and a storing instruction signal to image display apparatus 2 and image management apparatus 4. The apparatus setting data is data for setting an imaging condition or the like when imaging section 12 images an area to be inspected of the subject. The display setting data is setting data showing the contents of subject information displayed on the diagnostic screen in FIG. 6. Based on the display setting data, for example, the patient ID and name described above, and also sex, inspection history, age, area to be inspected, or the like can be displayed on the diagnostic screen. The subject setting data is a variable for setting current subject information such as the patient ID, and set every time the subject information is obtained from electronic medical record terminal 3. Setting data such as address data, apparatus setting data, or display setting data of electronic medical record terminal 3 to be coordinated can be changed by an input operation by the doctor etc., for example, on a setting screen (not shown) displayed in step S21.

Control section 11 reads the setting data, and then causes image processing section 13 to generate the initial screen in FIG. 6A and causes display section 14 to display the initial screen (step S22).

Control section 11 causes imaging section 12 to start imaging and causes image processing section 13 to start generation of an ultrasound image (step S23). More specifically, control section 11 outputs an imaging instruction signal to imaging section 12, causes imaging section 12 to scan the plurality of transducers in the ultrasound probe and start generation of two-dimensional data in frames on a brightness mode image and a color flow image. Then, control section 11 outputs an image generation instruction signal to image processing section 13, and causes image processing section 13 to start generation of ultrasound image data based on the two-dimensional data generated by imaging section 12. Control section 11 also outputs a display instruction signal to display section 14, and causes display section 14 to display an image of the ultrasound image data generated by image processing section 13.

As described above, when ultrasound diagnostic apparatus 1 according to this embodiment is powered on, imaging section 12 continuously performs imaging. Then, if the ultrasound probe of imaging section 12 is pressed against the area to be inspected of the subject, a taken ultrasound image is input to region T2 and displayed on the diagnostic screen of ultrasound diagnostic apparatus 1 (FIG. 6C).

Control section 11 first transmits to electronic medical record terminal 3 a signal to request subject information including identification information (patient ID) of the subject, and obtains currently set subject information from electronic medical record terminal 3 (step S24). Immediately after ultrasound diagnostic apparatus 1 is activated, the subject information is not set, but when the subject information is input to electronic medical record terminal 3, the subject information is automatically set without being input to ultrasound diagnostic apparatus 1. Control section 11 regularly obtains subject information from electronic medical record terminal 3 by poling. Thus, the subject information set in electronic medical record terminal 3 is sequentially reflected as the subject information in ultrasound diagnostic apparatus 1 according to this embodiment.

Then, control section 11 determines whether the subject (patient ID) obtained from electronic medical record terminal 3 has been changed from the currently set subject (patient ID) (step S25). If the patient ID obtained from electronic medical record terminal 3 is different from the currently set patient ID (step S25: YES), control section 11 changes subject information to change a subject to be inspected (step S26). On the other hand, if the obtained patient ID is the same as the currently set patient ID (step S25: NO), processing in step S27 is performed. For a first-visit patient, patient ID is not registered, and an inspection is performed without the patient ID being set.

In the subject information changing processing in step S26, control section 11 changes the currently set subject information (patient ID etc.) to the obtained subject information (patient ID etc.). Then, control section 11 causes image processing section 13 to update the diagnostic screen so that the changed subject information is displayed on the diagnostic screen (FIG. 6B).

In the next step, control section 11 determines whether operation input section 16 is operated (step S27). In this step, control section 11 determines whether any of operation buttons for an imaging stop operation, imaging restart operation, still image saving operation, moving image saving start operation, moving image saving finish operation, or the like provided as operation input section 16 is pressed. If any of the operation buttons is pressed, the indication is stored in a register as, for example, an identifiable operation signal and input through the register to control section 11.

Specifically, if operation input section 16 is not operated (step S27: NO), control section 11 returns to step S24 and continues the same processing as described above. If operation input section 16 is operated (step S27: YES), control section 11 performs conditional branching based on the operation signal, and performs processings corresponding to the imaging stop operation, imaging restart operation, still image saving operation, moving image saving start operation, and moving image saving finish operation (step S28 to S33). Then, after performing the corresponding processings, control section 11 returns to step S24 and continues the same processing as described above.

The processings corresponding to the imaging stop operation, imaging restart operation, still image saving operation, moving image saving start operation, and moving image saving finish operation when operation input section 16 is operated will be described (step S28 to S33).

When an unfreezing operation is performed, control section 11 causes imaging section 12 and image processing section 13 to restart operations that have been frozen by a freezing operation (step S28).

If the freezing operation is performed, control section 11 causes imaging section 12 and image processing section 13 to stop the operations (step S29). At this time, a frame buffer of image processing section 13 holds ultrasound image data last generated, and display section 14 stops updating an image with the ultrasound image data being input thereto. In other words, display section 14 displays a still image of the ultrasound image data last generated by image processing section 13.

If the still image saving operation is operated, control section 11 performs still image data saving processing (step S30).

FIG. 9 shows details of the still image data saving processing. In the still image data saving processing, control section 11 first outputs a save instruction signal to image processing section 13 and causes image processing section 13 to generate ultrasound image data for one frame to be saved (step S30a). Image processing section 13 generates the ultrasound image data to be saved, for example, by encoding into JPEG format image data for one frame when the button for the still image saving operation is pressed, and stores the ultrasound image data in memory section 17.

Next, control section 11 generates, for example, different image ID for each ultrasound image data to be saved as information for allowing identification of the ultrasound image data (step S30b). Then, control section 11 transmits to image management apparatus 4 the generated ultrasound image data of the still image to which the image ID is assigned and the storing instruction signal (step S30c). In this case, when patient ID is set, control section 11 transmits the patient ID assigned to the ultrasound image data to image management apparatus 4, and stores the image data in association with the current patient ID and image ID in the image data base of image management apparatus 4.

Then, control section 11 transmits the image ID and the display instruction signal to image display apparatus 2 (step S30d). As such, ultrasound diagnostic apparatus 1 transmits the storing instruction signal to image management apparatus 4 and the display instruction signal to image display apparatus 2, and thus both the apparatuses perform the processings in step S3 to S6 in FIG. 5.

Returning to the main processing loop in FIG. 8, steps S31 and S32 will be described. If the moving image saving start operation is performed, control section 11 starts recording moving image data (step S31). At this time, control section 11 causes image processing section 13 to start generation of moving image data to be saved. Image processing section 13 generates moving image data, for example, by encoding image data for a plurality of successive frames into MPEG format and stores the moving image data in memory section 17.

If the moving image saving finish operation is performed, control section 11 stops recording of the moving image data started by the moving image saving start operation (step S32). Then, as is the case for the still image data saving processing, control section 11 generates image ID, transmits the moving image data stored in memory section 17 and the image ID to image management apparatus 4, and stores the moving image data in the image data base of image management apparatus 4 (step S33).

After performing processings corresponding to these operations, control section 11 returns to step S24 and continues the same processing as described above. Control section 11 of ultrasound diagnostic apparatus 1 repeats the processings as described above to perform an inspection of the subject.

### Operation of Image Display Apparatus

Next, with reference to FIG. 10, details of an operation flow of image display apparatus 2 will be described.

FIG. 10 is a flowchart of an example of an operation of image display apparatus 2. The operation of image display apparatus 2 is an operation of control section 21, and realized, for example, by a CPU executing an image viewing program.

Control section 21 first performs initial setting to generate an ultrasound image viewing screen in FIG. 7 (step S41). In the initial setting, IP addresses or the like of ultrasound diagnostic apparatus 1 and image management apparatus 4 to be coordinated are set.

Image display apparatus 2 waits for receiving a display instruction signal from ultrasound diagnostic apparatus 1 (step S42: NO). If image display apparatus 2 has received the display instruction signal from ultrasound diagnostic apparatus 1, the display instruction signal is stored in a register included in image display apparatus 2 and identified by control section 21.

If image display apparatus 2 has received the display instruction signal from ultrasound diagnostic apparatus 1 (step S42: YES), image display apparatus 2 transmits to image management apparatus 4 identification information (image ID here) assigned to the display instruction signal and an image data request signal (step S43). Then, in response to receiving corresponding ultrasound image data from image management apparatus 4, image display apparatus 2 displays an image of the ultrasound image data on the ultrasound image viewing screen (step S44). After displaying the image of the ultrasound image data on the ultrasound image viewing screen, image display apparatus 2 returns to step S42, and waits for receiving the display instruction signal from ultrasound diagnostic apparatus 1.

Image display apparatus 2 also desirably receives subject information from ultrasound diagnostic apparatus 1 or electronic medical record terminal 3. For example, when receiving the display instruction signal from ultrasound diagnostic apparatus 1, image display apparatus 2 receives the image ID and the subject information (for example, patient ID). Then, image display apparatus 2 can initialize the ultrasound image viewing screen every time the subject is changed so that the ultrasound image of the subject before change is not displayed, without requiring an operation by the doctor etc.

### Operation of Image Management Apparatus

Next, with reference to FIG. 11, details of an operation flow of image management apparatus 4 will be described.

FIG. 11 is a flowchart of an example of an operation of image management apparatus 4. The operation of image management apparatus 4 is an operation of a control section (not shown) of image management apparatus 4, and is realized, for example, by a CPU executing an image management program.

Image management apparatus 4 determines whether it has received a storing instruction signal of ultrasound image data from ultrasound diagnostic apparatus 1 (step S51). Then, if image management apparatus 4 has received the storing instruction signal (step S51: YES), the received ultrasound image data is stored in the image data base in association with image ID assigned to the storing instruction signal (step S52). If image management apparatus 4 has received the storing instruction signal from ultrasound diagnostic apparatus 1, the storing instruction signal is stored in a register included in image management apparatus 4, and identified by the control section of image management apparatus 4.

If image management apparatus 4 has not received the storing instruction signal from ultrasound diagnostic apparatus 1 (step S51: NO), image management apparatus 4 determines whether it has received an image data request signal from image display apparatus 2 (step S53). Then, if image management apparatus 4 has received the image data request signal (step S53: YES), image management apparatus 4 transmits ultrasound image data of corresponding image ID to image display apparatus 2 (step S54). If image management apparatus 4 has not received the image data request signal (step S53: NO), image management apparatus 4 returns to step S51 and again continues monitoring processing.

As described above, in the ultrasound diagnostic system according to this embodiment, the ultrasound image can be displayed by image display apparatus 2 without requiring an operation on image display apparatus 2. In other words, ultrasound diagnostic apparatus 1 causes image management apparatus 4 to store the identification information such as the image ID assigned to the ultrasound image data, and provides image display apparatus 2 an instruction to display the ultrasound image data together with the identification information, thereby allowing ultrasound diagnostic apparatus 1 to independently automatically control both the apparatuses. Thus, the doctor etc. does not need to search corresponding data from image management apparatus 4 when viewing the ultrasound image on image display apparatus 2 after imaging the ultrasound image using ultrasound diagnostic apparatus 1.

In particular, in the ultrasound diagnostic system according to this embodiment, ultrasound diagnostic apparatus 1 generates the information for identifying the ultrasound image such as the image ID to cause image management apparatus 4 and image display apparatus 2 to operate. Then, even for a first-visit patient without patient ID and the like, image management apparatus 4 can identifiably save ultrasound image data, and image display apparatus 2 can obtain the ultrasound image data. Thus, the doctor etc. does not need to perform different saving operations for different patients such that the doctor etc. prepares a new medical record and then saves ultrasound image data for a first-visit patient and saves ultrasound image data in association with an existing medical record for a return-visit patient. This allows the doctor etc. to use ultrasound diagnostic apparatus 1 without any burden, like using a stethoscope.

### Variant

In the embodiment described above, ultrasound diagnostic apparatus 1 transmits the display instruction signal of the ultrasound image to image display apparatus 2 every time the still image saving operation is performed (save instruction signal is generated). However, ultrasound diagnostic apparatus 1 may be configured so that condition setting in transmission of the display instruction signal is performed.

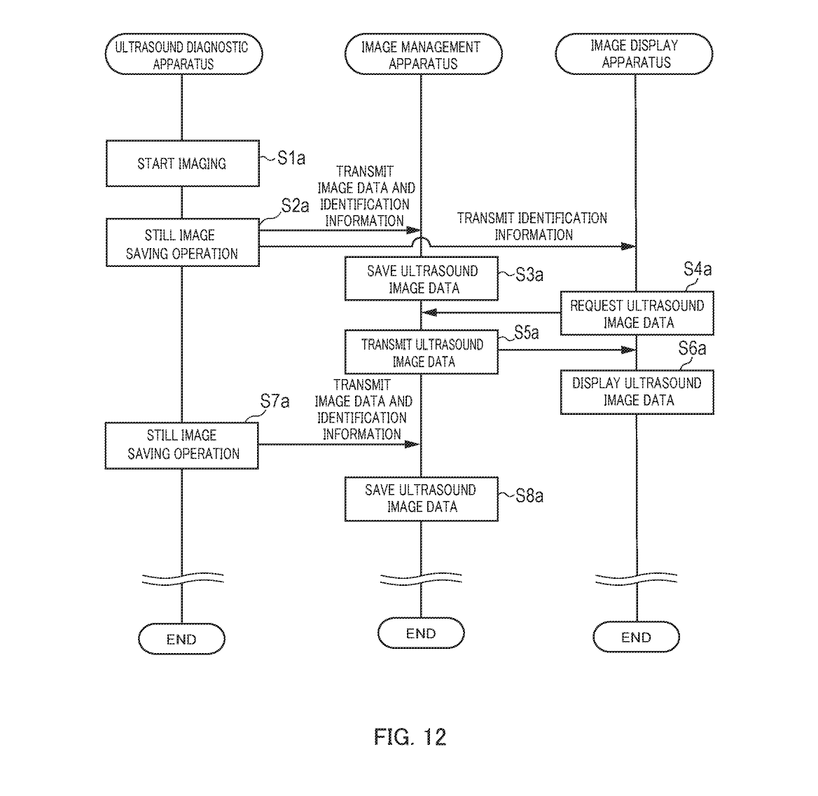

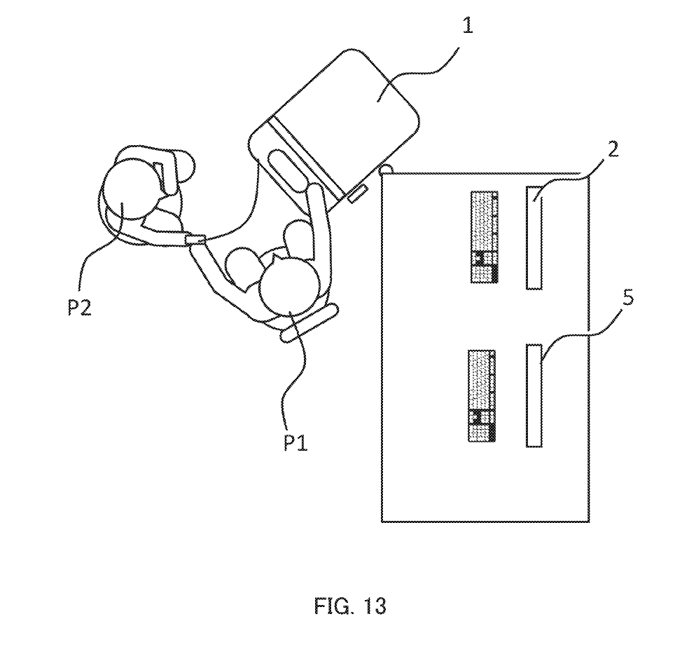

FIG. 12 shows an example of an operation flow of an ultrasound diagnostic system according to this variant. FIG. 12 is different from FIG. 5 in only an operation of ultrasound diagnostic apparatus 1 in step S7a when a second still image saving operation is performed for an ultrasound image of the same subject. Specifically, when the still image saving operation is performed, ultrasound diagnostic apparatus 1 transmits a storing instruction signal to image management apparatus 4, but does not transmit a display instruction signal to image display apparatus 2. Other operations in steps S1a to S7a, S8a to S9a are the same as those in steps S1 to S7 or the like in FIG. 5, and descriptions thereof will be omitted.

To achieve such a function, ultrasound diagnostic apparatus 1 includes, as setting data, data on condition setting for determining whether the display instruction signal is transmitted, and setting for defining a timing of transmitting the display instruction signal. Control section 11 of ultrasound diagnostic apparatus 1 determines an output mode of the display instruction signal based on the setting data.

For example, ultrasound diagnostic apparatus 1 determines whether it transmits the display instruction signal based on the setting data, and does not transmit the display instruction signal if the number of still image saving operations is a predetermined number or more or in an operation of saving moving image data. In this case, control section 11 of ultrasound diagnostic apparatus 1 may be configured to store the number of still image saving operations for an ultrasound image of the same subject in memory section 17, and increment the number for each still image saving operation.

Then, in the case where the ultrasound image viewing screen of image display apparatus 2 can display only one ultrasound image, or the like, frequent switching of the ultrasound image displayed on the ultrasound image viewing screen every time the ultrasound image is saved can be prevented. In other words, the doctor etc. can perform an examination while viewing a first obtained ultrasound image.

Ultrasound diagnostic apparatus 1 can determine a timing of transmission of the display instruction signal based on an image type of image data to be saved. For example, when causing image display apparatus 2 to display moving image data or the like, ultrasound diagnostic apparatus 1 can set so that a timing of output of the display instruction signal is delayed by a certain time from a timing of transmission of the storing instruction signal to image management apparatus 4. If image management apparatus 4 stores the moving image data, it may take a certain time from when ultrasound diagnostic apparatus 1 transmits the storing instruction signal to image management apparatus 4 until storage of the moving image data in image management apparatus 4 is completed because of a large amount of data. Thus, the timing of transmission of the display instruction signal is delayed by a certain time from the timing of transmission of the storing instruction signal from ultrasound diagnostic apparatus 1 to image management apparatus 4, thereby reducing a waiting time in image display apparatus 2 after transmission of the image data request signal to image management apparatus 4 until obtaining corresponding image data from image management apparatus 4.

### Other Embodiments

The present invention is not limited to the embodiment described above, but variations are considered.

In the embodiment described above, the image ID is taken as an example of the “identification information” assigned to the ultrasound image data by ultrasound diagnostic apparatus 1. However, the identification information does not always need to be the image ID as long as it is information for allowing identification of ultrasound image data, and may be examination ID indicating the order of examination, or the like. The same examination ID is used for the same subject, and when a plurality of ultrasound image data are saved, image management apparatus 4 and image display apparatus 2 may identify the plurality of ultrasound image data based on the examination ID and an instruction signal receiving time from ultrasound diagnostic apparatus 1.

Although, in the embodiment described above, the timing when the doctor etc. saves the still image of the ultrasound image is taken as an example of a timing of generation of the “save instruction signal”, the opportunity may vary as long as it concerns operation or the like that is performed by ultrasound diagnostic apparatus 1 to cause image management apparatus 4 to store ultrasound image data. For example, if a scan start operation is required, a save instruction signal may be generated at certain intervals when the scan start operation is performed. If image display apparatus 2 is caused to display ultrasound image data of a moving image, a timing when the moving image saving finish operation is performed may be used.

In the embodiment described above, as an example of a configuration of the ultrasound diagnostic system, image display apparatus 2 and electronic medical record terminal 3 are separate, but image display apparatus 2 and electronic medical record terminal 3 may be integrated. In other words, image display apparatus 2 having a function program of electronic medical record terminal 3 may be used.

Specific examples of the present invention have been described in detail, but they are merely exemplary and do not limit the scope of claims. Technologies defined in the appended claims may include variations and changes of the specific examples described above.

The descriptions of the specification and accompanying drawings reveal at least the following matters.

Ultrasound diagnostic apparatus 1 configured to be able to communicate with image display apparatus 2 that displays an ultrasound image of an area to be inspected of a subject and image management apparatus 4 that manages image data of the ultrasound image is disclosed, including: input section 16 that receives an operation input by an operator; and control section 11 that transmits the image data of the ultrasound image and identification information to image management apparatus 4 and causes image management apparatus 4 to store the image data in association with the identification information according to a save instruction signal generated based on the operation input, and further transmits to image display apparatus 2 the identification information and a display instruction signal for causing image display apparatus 2 to display the image data of the ultrasound image and causes image display apparatus 2 to obtain the image data of the ultrasound image from image management apparatus 4.

The identification information may be generated by control section 11.

Control section 11 may not transmit the display instruction signal to image display apparatus 2 when the number of the save instruction signals for the same subject is a predetermined number or more.

Control section 11 may determine a timing of transmission of the display instruction signal based on an image type of the image data.

The save instruction signal may be generated according to an input of an operation of saving a still image of the ultrasound image.

An ultrasound diagnostic program to be executed by ultrasound diagnostic apparatus 1 configured to be able to communicate with image display apparatus 2 that displays an ultrasound image of an area to be inspected of a subject and image management apparatus 4 that manages image data of the ultrasound image is disclosed, including: a process of receiving an operation input by an operator; and a process of transmitting the image data of the ultrasound image and identification information to image management apparatus 4 and causing image management apparatus 4 to store the image data in association with the identification information according to a save instruction signal generated based on the operation input, and further transmitting to image display apparatus 2 the identification information and a display instruction signal for causing image display apparatus 2 to display the image data of the ultrasound image and causing image display apparatus 2 to obtain the image data of the ultrasound image from image management apparatus 4.

The contents of disclosure of the specification, drawings, and abstract of Japanese Patent Application Laid-Open No. 2016-063517 filed on Mar. 28, 2016 are entirely incorporated herein.

### INDUSTRIAL APPLICABILITY

The present disclosure may be favorably applied to a medical diagnostic apparatus of a medical diagnostic system.

### REFERENCE SIGNS LIST

1 ultrasound diagnostic apparatus

2 image display apparatus

3 electronic medical record terminal

4 image management apparatus

5 electronic medical record management apparatus

6 communication line

1. A medical diagnostic apparatus configured to be capable of communicating with an image display apparatus that displays a medical image of an area to be inspected of a subject and an image management apparatus that manages image data of the medical image, comprising:
a hardware processor that receives an operation input by an operator and
transmits the image data of the medical image and identification information to the image management apparatus and causes the image management apparatus to store the image data in association with the identification information according to a save instruction signal generated based on the operation input, and
further transmits to the image display apparatus the identification information and a display instruction signal for causing the image display apparatus to display the image data of the medical image and causes the image display apparatus to obtain the image data of the medical image from the image management apparatus.

2. The medical diagnostic apparatus according to claim 1, wherein the identification information is generated by the hardware processor.

3. The medical diagnostic apparatus according to claim 1, wherein the hardware processor does not transmit the display instruction signal to the image display apparatus when the number of the save instruction signals for the same subject is a predetermined number or more.

4. The medical diagnostic apparatus according to claim 1, wherein the hardware processor determines a timing of transmission of the display instruction signal based on an image type the image data.

5. The medical diagnostic apparatus according to claim 1, wherein the save instruction signal is generated according to an input of an operation of saving a still image of the medical image.

6. The medical diagnostic apparatus according to claim 1, wherein the medical diagnostic apparatus is applied to an ultrasound diagnostic apparatus that generates an ultrasound image as the medical image.

7. A non-transitory computer-readable recording medium that stores a diagnostic program to be executed by a medical diagnostic apparatus configured to be capable of communicating with an image display apparatus that displays a medical image of an area to be inspected of a subject and an image management apparatus that manages image data of the medical image, the diagnostic program comprising:
a process of receiving an operation input by an operator; and
a process of transmitting the image data of the medical image and identification information to the image management apparatus and causing the image management apparatus to store the image data in association with the identification information according to a save instruction signal generated based on the operation input, and
further transmitting to the image display apparatus the identification information and a display instruction signal for causing the image display apparatus to display the image data of the medical image and causing the image display apparatus to obtain the image data of the medical image from the image management apparatus.
